(19) United States
(12) Patent Application Publication
(10) Pub. No.: US 2002/0192741 A1
(43) Pub. Date: Dec. 19, 2002

(54) Selective medium for gram-positive bacteria

(76) Inventor: John L. McKillip, Ruston, LA (US)

Correspondence Address: R. Keith Harrison, 2139 E. Bert Kouns, Shreveport, LA, 71105, US

(21) Appl. No.: 10/144,690

(22) Filed: May 13, 2002

Related U.S. Application Data
(63) Continuation-in-part of application No. 09/676,606, filed on Sep. 28, 2000, now abandoned.

(51) Int. Cl. C12Q 1/04; C12N 1/20

(52) U.S. Cl. 435/34; 435/252.1

(57) ABSTRACT

A selective and differential medium for Gram-positive bacteria. In a preferred embodiment, the medium is characterized by a Gram-positive agar including appropriate quantities of sodium chloride, sodium glycerophosphate, lithium chloride, phenylethanol, mannitol, tryptone, yeast extract, bromcresol purple and agar. When streaked with mixed bacterial cultures containing both Gram-positive and Gram-negative bacteria, the agar promotes growth and colonization of the Gram-positive bacteria and inhibits growth of the Gram-negative bacteria in the culture. The pH indicator bromcresol purple facilitates differentiation of Gram-positive species which ferment the sugar mannitol in the agar, such as Staphylococcus aureus, from those which do not, such as Staphylococcus epidermis. In another embodiment, the medium is characterized by a Gram-positive broth including sodium chloride, sodium glycerophosphate, lithium chloride, phenylethanol, mannitol, tryptone, yeast extract and bromcresol purple.

### CROSS-REFERENCE TO RELATED APPLICATIONS

This is a continuation-in-part of application Ser. No. 09/676,606, filed Sep. 28, 2000.

### BACKGROUND OF THE INVENTION

1. Field of the Invention

This invention relates to selective growth media for microorganisms and more particularly, to a selective and differential medium for Gram-positive bacteria, which medium in a preferred embodiment is characterized by a Gram-positive agar including appropriate quantities of sodium chloride, sodium glycerophosphate, lithium chloride, phenylethanol, mannitol, tryptone, yeast extract, bromcresol purple and agar. When streaked with mixed bacterial cultures containing both Gram-positive and Gram-negative bacteria, the agar promotes growth and colonization of Gram-positive bacteria and inhibits growth of Gram-negative bacteria in the culture. The pH indicator bromcresol purple enables differentiation of those species of Gram-positive bacteria which ferment the sugar mannitol, such as Staphylococcus aureus, from those which do not, such as Staphylococcus epidermis. In a second embodiment, the medium is characterized by a Gram-positive broth including sodium chloride, sodium glycerophosphate, lithium chloride, phenylethanol, mannitol, tryptone, yeast extract and bromcresol purple.

In a typical undergraduate Introductory Microbiology laboratory course, students undertake a project in which they are given a broth tube containing a mixture of three bacteria of unknown genus and species and are required to implement an array of biochemical and morphological tests to separate and identify the types of bacteria from the tube. The project gives the students a chance to apply the procedures presented in the laboratory up to the time of the project, as well as an opportunity to practice their techniques and reasoning when presented with a typical microbiology problem. The students enjoy the benefits of working on their own problems and learning from any mistakes they make.

Having completed the typical battery of biooxidation and hydrolysis tests a few weeks earlier in the course, and familiar with the “how” and “why” of each of these tests, the students are allowed to use a variety of selective and differential media to first isolate each of their three bacteria in the mixed culture. Only after a confirmatory Gram stain to verify the purity of each isolate are they allowed to proceed with their biochemical tests and morphological observations to make their final determination as to the identity of each of the three unknown bacterial types.

Although a properly-performed culture streak is usually adequate to isolate a mixture of two or even three different bacterial species on a generic medium such as trypticase soy agar, the students are encouraged to use selective media (a medium that allows some microorganisms but not others to grow) to assist them in separating the Gram-negative bacteria from the Gram-positive bacteria, and vice-versa. For Gram-negative isolation, a streak of the mixed culture on MacConkey's agar is usually sufficient, and this procedure additionally allows the student to observe possible lactose fermentation by some species of the Gram-negative bacteria, indicated by a change in color of the isolated colonies (a red color indicates positive lactose fermentation). The high degree of selectivity for Gram-negative bacteria renders MacConkey's agar a dependable medium for obtaining a pure culture of the Gram-negative bacteria from a mixed culture.

For isolation and colonization of Gram-positive bacteria and selection against Gram-negative bacteria, phenylethanol agar (PEA) plates are typically used. PEA plates, however, are ineffective in this regard since several species of Gram-negative bacteria, including E. coli, grow quite well on the plates. Moreover, colonization and growth of some Gram-positive bacteria, such as Bacillus spp., is completely inhibited on the medium. Another type of Gram-positive medium, Mannitol Salt Agar (MSA), promotes growth of some Gram-positive bacteria such as staphylococci but tends to select against most other Gram-positive bacteria such as Bacillus spp.

U.S. Pat. No. 6,051,394, dated Apr. 18, 2000, to Simmons, et al., describes “Detection of Microorganisms”, characterized by a method and kit for detecting microorganisms in a mass or liquid, or on a surface. The method is conducted by obtaining a sample of microorganisms and placing the sample in a sterile chamber and in contact with a composition adapted to detect microorganisms in the sample. The sterile chamber may contain a filter for isolating any microorganisms present in the sample. Sterile sampling instruments, a delineating instrument and a mechanism for typing the microorganisms may also be provided.

An object of the present invention is to provide a new and improved bacterial culture medium which selects for Gram-positive bacteria and against Gram-negative bacteria.

Another object of this invention is to provide a new and improved, selective and differential bacterial culture medium characterized by a Gram-positive agar.

Still another object of the invention is to provide a new and improved, selective and differential bacterial culture medium characterized by a Gram-positive broth.

Yet another object of the invention is to provide a selective medium for Gram-positive bacteria, characterized by a carbon source, a vitamin and mineral source and one or a selected combination of agents which promote growth and colonization of Gram-positive bacteria and inhibit growth and colonization of Gram-negative bacteria in the medium.

Still another object of this invention is to provide a selective medium for Gram-positive bacteria, which medium includes a pH indicator for indicating sugar fermentation and thus, the presence of certain species of Gram-positive bacteria on the medium.

A still further object of this invention is to provide a selective and differential medium for Gram-positive bacteria, characterized by a Gram-positive agar which includes appropriate quantities of sodium chloride, sodium glycerophosphate, lithium chloride, phenylethanol, mannitol, tryptone, yeast extract, bromcresol purple and agar.

Yet another object of this invention is to provide a selective and differential medium for Gram-positive bacteria, characterized by a Gram-positive broth including sodium chloride, sodium glycerophosphate, lithium chloride, phenylethanol, mannitol, tryptone, yeast extract and bromcresol purple.

### SUMMARY OF THE INVENTION

These and other objects of the invention are provided in a new and improved, selective and differential medium for Gram-positive bacteria. In a preferred embodiment, the medium is characterized by a Gram-positive agar including appropriate quantities of sodium chloride, sodium glycerophosphate, lithium chloride, phenylethanol, mannitol, tryptone, yeast extract, bromcresol purple and agar. When streaked with mixed bacterial cultures containing both Gram-positive and Gram-negative bacteria, the agar promotes growth and colonization of Gram-positive bacteria and inhibits growth of Gram-negative bacteria in the culture. The pH indicator bromcresol purple facilitates differentiation of Gram-positive bacterial species which ferment the sugar mannitol in the agar, such as Staphylococcus aureus, from those which do not, such as Staphylococcus epidermis. In another embodiment, the medium is characterized by a Gram-positive broth including sodium chloride, sodium glycerophosphate, lithium chloride, phenylethanol, mannitol, tryptone, yeast extract and bromcresol purple.

### DESCRIPTION OF THE PREFERRED EMBODIMENTS

In a preferred embodiment, the selective and differential medium for Gram-positive bacteria of this invention is typically characterized by appropriate quantities of sodium chloride, sodium glycerophosphate, lithium chloride, phenylethanol, mannitol, tryptone, yeast extract, bromcresol purple and agar to form an agar which selects for growth and colonization of Gram-positive bacteria and against growth and colonization of Gram-negative bacteria. The sodium chloride, lithium chloride and phenylethanol act as selective agents which promote the growth and colonization of Gram-positive bacteria while inhibiting the growth of Gram-negative bacteria, and the sodium glycerophosphate is a buffering agent. The mannitol sugar is a carbon source for the bacteria, and the tryptone and the yeast extract are vitamin and mineral sources. The bromcresol purple is a pH indicator which turns the normal purple color of the agar to a yellow color in the presence of mannitol fermentation. Accordingly, the transition in color facilitates distinguishing between the Gram-positive bacteria Staphylococcus aureus, which is positive for mannitol fermentation, from the Gram-positive bacteria Staphylococcus epidermis, which is negative for mannitol fermentation. Mannitol fermentation is also useful for differentiating members of the family Bacillaceae. In a second embodiment, the selective and differential medium for Gram-positive bacteria is characterized by a Gram-positive broth typically including sodium chloride, sodium glycerophosphate, lithium chloride, phenylethanol, mannitol, tryptone, yeast extract and bromcresol purple. Typical w/v concentrations for the sodium chloride, lithium chloride and phenylethanol in both the agar and the broth are about 3.2% to about 3.8% sodium chloride, about 1.0% to about 1.5% lithium chloride, and about 0.15% to about 0.19% phenylethanol.

A typical 1-liter batch of the Gram-positive agar of this invention is typically prepared as follows. About 32 grams of sodium chloride, about 5 grams of sodium glycerophosphate, about about 10 grams of lithium chloride, about 1.5 mL of phenylethanol about 15 grams of mannitol, about 10 grams of tryptone, about 10 grams of yeast extract and about 0.02 grams of bromcresol purple are dissolved in about 1 liter of deionized water as the water is stirred and heated to form a substantially homogenous solution. As stirring and heating is continued, about 15 grams of agar is added to the solution. After it is brought to a gentle boil, the solution is removed from heat. Finally, the solution is autoclaved and poured into multiple Petri dishes.

The components of the typical 1-liter Gram-positive agar batch are summarized in Table 1:

TABLE 1

| Gram-positive agar components | Component quantities, per liter |
|---|---|
| Sodium Chloride | 32 g |
| Sodium glycerophosphate | 5 g |
| Lithium chloride | 10 g |
| Phenylethanol | 1.5 mL |
| Mannitol | 15 g |
| Tryptone | 10 g |
| Yeast extract | 10 g |
| Bromcresol purple | 0.02 g |
| Agar | 15 g |

In typical application of the Gram-positive agar, a mixed bacterial culture containing both Gram-positive and Gram-negative bacteria is streaked on the surface of the agar according to conventional inoculation techniques, and the streaked agar is incubated at about 30° C. or 37° C. typically for about 96 hours. Growth and colonization is negative for each species of Gram-negative bacteria inoculated on the agar, whereas growth and colonization is positive for each species of Gram-positive bacteria. Mannitol fermentation by some species of Gram-positive bacteria is indicated by a change in agar color, from the normal purple to a yellowish color.

Table 2 summaries the growth characteristics of selected Gram-positive and Gram-negative bacteria streaked on the GP agar:

TABLE 2
Growth characteristics of selected bacteria streaked on GP agar

| | Incubation | Growth 48 hours | Mannitol |
|---|---|---|---|
| Organism | Temperature | after incubation | Fermentation |
| Escherichia coli | 37° C. | no | NA¹ |
| strain B (GN)⁵ | | | |
| Escherichia coli | 37° C. | no | NA |
| strain INV ∝′ (GN) | | | |
| Salmonella | 37° C. | no | NA |
| typhimurium (MSU²) | | | |
| (GN) | | | |
| Salmonella | 37° C. | no | NA |
| typhimurium ATCC | | | |
| 14028³ (GN) | | | |
| Shigella flexneri | 37° C. | no | NA |
| (MSU) (GN) | | | |
| Shigella sonnei | 37° C. | no | NA |
| (MSU) (GN) | | | |
| Proteus vulgaris | 37° C. | no | NA |
| (MSU) (GN) | | | |
| Citrobacter freundii | 37° C. | no | NA |
| (MSU) (GN) | | | |
| Serratia marcescens | 37° C. | no | NA |
| (MSU) (GN) | | | |
| Enterobacter | 37° C. | no | NA |
| aerogenes (MSU) | | | |
| (GN) | | | |
| Enterobacter cloacae | 37° C. | no | NA |
| (MSU) (GN) | | | |
| Pseudomonas | 37° C. | no | NA |
| aeruginosa ATCC 27853 | | | |
| (GN) | | | |
| Staphylococcus | 37° C. | yes | yes |
| aureus ATCC 13565 | | | |
| (GP)⁶ | | | |
| Staphylococcus | 37° C. | yes | no |
| epidermis (MSU) | | | |
| (GP) | | | |
| Streptococcus | 37° C. | yes | no |
| pyogenes ATCC 19615 | | | |
| (GP) | | | |
| Leuconostoc | 30° C. | yes | no |
| cremoris (MSU) | | | |
| (GP) | | | |
| Bifidobacterium | 37° C. | yes | no |
| longum (MSU) (GP) | | | |
| Bacillus megaterium | 30° C. | yes | yes |
| strain B4A (GP) | | | |
| Bacillus cereus | 30° C. | yes | no |
| (WSU⁴) (GP) | | | |
| Bacillus cereus ATCC | 30° C. | yes | no |
| 14579 (GP) | | | |
| Bacillus subtilis | 30° C. | yes | no |
| strain B6A (GP) | | | |
| Bacillus | 30° C. | yes | yes |
| licheniformis ATCC | | | |
| 14580 (GP) | | | |
| Listeria | 37° C. | yes | no |
| monocytogenes ATCC | | | |
| 43256 (GP) | | | |

¹Not applicable
²MSU (strain obtained from teaching stocks at Mississippi State University, Department of Biology).
³ATCC (strain obtained from American Type Culture Collection, Rockville, MD).
⁴WSU (stain obtained from teaching stocks at Washington State University, Department of Microbiology).
⁵Gram-negative bacterium
⁶Gram-positive bacterium

A typical 1-liter batch of the Gram-positive broth of this invention is typically prepared as follows. About 32 grams of sodium chloride, about 5 grams of sodium glycerophosphate, about about 10 grams of lithium chloride, about 1.5 mL of phenylethanol, about 15 grams of mannitol, about 10 grams of tryptone, about 10 grams of yeast extract and about 0.02 grams of bromcresol purple are dissolved in about 1 liter of deionized water as the water is heated and stirred to form a substantially homogenous solution. The solution is brought to a gentle boil as stirring continues, and then removed from heat. Finally, the solution liquid is autoclaved and poured into test tubes.

The components of the typical 1-liter Gram-positive broth batch are summarized in Table 3:

TABLE 3

| Gram-positive broth components, per liter | Component quantities |
|---|---|
| Sodium chloride | 32 g |
| Sodium glycerophosphate | 5 g |
| Lithium chloride | 10 g |
| Phenylethanol | 1.5 mL |
| Mannitol | 15 g |
| Tryptone | 10 g |
| Yeast extract | 10 g |
| Bromcresol purple | 0.02 g |

In typical application of the Gram-positive broth, the broth is inoculated with a mixed bacterial culture containing both Gram-positive and Gram-negative bacteria according to conventional inoculation techniques, and incubated at typically about 30° C. or 37° C. for about 96 hours. Growth is negative for each species of Gram-negative bacteria inoculated in the broth, whereas growth is positive for each species of Gram-positive bacteria inoculated in the broth. Mannitol fermentation by some species of the Gram-positive bacteria is indicated by a change in agar color, from the normal purple to a yellowish color. The growth characteristics of selected bacteria inoculated in the GP broth mimics those characteristics observed for the GP agar (Table 2).

The invention will be better understood by consideration of the following examples:

### EXAMPLE 1

A 1-liter batch of Gram-positive agar was prepared by initially dissolving about 32 g of sodium chloride, about 5 g of sodium glycerophosphate, about 10 g g of lithium chloride, about 1.5 mL of phenylethanol, about 15 g of mannitol, about 10 g of tryptone, about 10 g of yeast extract and about 0.02 g of bromcresol purple in about 1 liter of water while heating and stirring the water. About 15 g of agar was added to the water and stirred to form a substantially homogenous mixture, and the mixture was briefly brought to a gentle boil as stirring continued. The mixture was removed from heat, autoclaved and poured into multiple Petri dishes.

### EXAMPLE 2

A 1-liter batch of Gram-positive broth was prepared by initially dissolving about 32 g of sodium chloride, about 5 g of sodium glycerophosphate, about 10 g of lithium chloride, about 1.5 mL of phenylethanol, about 15 g of mannitol, about 10 g of tryptone, about 10 g of yeast extract and about 0.02 g of bromcresol purple in about 1 liter of water while stirring and heating the water to form a substantially homogenous mixture. The mixture was brought to a gentle boil, removed from heat, autoclaved and poured into multiple test tubes.

### EXAMPLE 3

A mixed bacterial culture containing both Gram-positive and Gram-negative bacteria was streaked on the GP agar prepared according to the method of Example 1, and the GP agar was then incubated for about 48 hours. All species of Gram-positive bacteria from the mixed bacterial culture were observed growing in colonies on the agar, while none of the Gram-negative bacteria from the mixed culture was observed growing on the agar. Mannitol-fermenting species of the Gram-positive bacteria was indicated by a color change in the agar from purple to yellow.

### EXAMPLE 4

A test tube containing Gram-positive broth prepared according to the method of Example 2, was inoculated with a mixed bacterial culture containing both Gram-positive and Gram-negative bacteria, and the inoculated broth was incubated for about 48 hours. All species of Gram-positive bacteria from the culture were observed growing in colonies in the broth, while none of the Gram-negative bacteria was observed growing in the broth. Mannitol-fermenting species of Gram-positive bacteria was indicated by a color change in the broth from purple to yellow.

It will be appreciated by those skilled in the art that the selective medium for Gram-positive bacteria of this invention is useful for selectively promoting separation of those species of Gram-positive and Gram-negative bacteria which are typically used in mixed culture experiments in undergraduate Introductory Microbiology laboratory courses, by promoting growth and colonization of the Gram-positive bacteria while inhibiting growth of the Gram-negative bacteria. It is understood that various carbon sources other than mannitol can be used, such as sucrose, fructose, sorbitol, galactose, maltose, erythritol, ethyleneglycol, ethanol, in non-exclusive particular. Alternatively, sucrose may be combined with dextrin hydrolysate, citrus molasses, beet molasses, squeezed juice from beet or sugar cane or juice from citrus, in non-exclusive particular. It is further understood that various other pH indicators other than bromcresol purple such as phenyl red and bromphenyl blue, in non-exclusive particular, can be used to indicate the presence of mannose-fermenting Gram-positive bacteria in the Gram-positive agar or broth. While each of the sodium chloride, sodium glycerophosphate and phenylethanol can be used alone or in combination with one of the others in the Gram-positive agar or broth to facilitate some degree of selection against Gram-negative bacteria, these components exhibit their maximal selecting effect when used in combination with each other as described above in Examples 1 and 2. Other buffering agents capable of use instead of the sodium glycerophosphate in the Gram-positive agar or broth include exchange resins, water-soluble trisodium citrate, disodium citrate, ammonium citrate dibasic, sodium glycerophosphate, alkali metals and alkaline earth metal bases or salts, in non-exclusive particular. Furthermore, various other nitrogen, vitamin and mineral sources other than tryptone and yeast extract which are known to those skilled in the art and suitable for sustaining growth and colonization of gram-positive bacteria, can be used for the purpose.

While the preferred embodiments of the invention have been described above, it will be recognized and understood that various modifications can be made in the invention and the appended claims are intended to cover all such modifications which may fall within the spirit and scope of the invention.

Having described my invention with the particularity set forth above, what is claimed is:

1. A method of selecting for Gram-positive bacteria and against Gram-negative bacteria in a mixed bacterial culture, said method comprising:
(a) preparing a selective medium comprising sodium chloride, lithium chloride, phenylethanol, a buffering agent, a carbon source and a vitamin and mineral source;
(b) inoculating said selective medium with the mixed bacterial culture; and
(c) incubating said selective medium for a period of time.

2. The method of claim 1 wherein said selective medium comprises agar.

3. The method of claim 1 wherein said selective medium comprises a pH indicator for indicating bacterial fermentation of said carbon source.

4. The method of claim 3 wherein said selective medium comprises agar.

5. The method of claim 1 wherein said selective medium comprises broth.

6. The method of claim 5 wherein said broth comprises a pH indicator for indicating bacterial fermentation of said carbon source.

7. The method of claim 1 wherein said carbon source comprises mannitol.

8. The method of claim 7 wherein said selective medium comprises agar.

9. The method of claim 7 wherein said selective medium comprises a pH indicator for indicating bacterial fermentation of said mannitol.

10. The method of claim 9 wherein said selective medium comprises agar.

11. The method of claim 7 wherein said selective medium comprises broth.

12. The method of claim 11 wherein said selective medium comprises a pH indicator for indicating bacterial fermentation of said mannitol.

13. A method of selecting for Gram-positive bacteria and against Gram-negative bacteria in a mixed bacterial culture, said method comprising:
(a) preparing a selective medium comprising about 3.2% to about 3.8% (w/v) sodium chloride, about 1.0% to about 1.5% (w/v) lithium chloride, about 0.15% to about 0.19% (w/v) phenylethanol, a buffering agent, a carbon source and a vitamin and mineral source;
(b) inoculating said selective medium with the mixed bacterial culture; and
(c) incubating said selective medium for about 24 to about 72 hours at a temperature of about 30° C. to about 37° C.

14. The method of claim 13 wherein said carbon source comprises mannitol.

15. The method of claim 13 wherein said selective medium comprises a pH indicator for indicating bacterial fermentation of said carbon source.

16. The method of claim 15 wherein said carbon source comprises mannitol.

17. The method of claim 13 wherein said buffering agent comprises sodium glycerophosphate.

18. The method of claim 17 wherein said selective medium comprises a pH indicator for indicating bacterial fermentation of said carbon source.

19. A method of selecting for Gram-positive bacteria and against Gram-negative bacteria in a mixed bacterial culture, said method comprising:
(a) preparing a selective medium comprising about 3.2% to about 3.8% (w/v) sodium chloride, about 1.0% to about 1.5% (w,v) lithium chloride, about 0.15% to about 0.19% (w/v) phenylethanol, a buffering agent, a carbon source and a vitamin and mineral source;
(b) inoculating said selective medium with the mixed bacterial culture; and
(c) incubating said selective medium for about 96 hours at a temperature of about 30° C. to about 37° C.

20. The method of claim 19 wherein said selective medium comprises a pH indicator for indicating bacterial fermentation of said carbon source.
